(19) United States
(12) Patent Application Publication
(10) Pub. No.: US 2004/0014696 A1
(43) Pub. Date: Jan. 22, 2004

(54) Specificity in treatment of diseases

(76) Inventors: Johnson Lau, Newport Beach, CA (US); Zhi Hong, Aliso Viejo, CA (US); Chin-chung Lin, Irvine, CA (US)

Correspondence Address: ROBERT D. FISH; RUTAN & TUCKER, LLP, P.O. BOX 1950, 611 ANTON BLVD., 14TH FLOOR, COSTA MESA, CA, 92628-1950, US

(21) Appl. No.: 10/343,529

(22) PCT Filed: Dec. 7, 2000
(86) PCT No.: PCT/US00/33454

(51) Int. Cl. A61K 31/7056; A61K 31/52

(52) U.S. Cl. 514/43; 514/263.1

(57) ABSTRACT

A drug is modified by covalently coupling a blocking group to the drug via a nitrogen atom in the blocking group. The blocking group in the modified drug prevents metabolic conversion and sequestration of the drug in non-target cells, and the blocking group is enzymatically removed in the target cell. Particularly contemplated advantages of presented compounds and methods include reduction of cytotoxicity by inhibition of metabolic conversion to potentially cytotoxic metabolites, reduction of dosages of drugs by reduction of sequestration into non-target cells, and increase of selectivity of a drug.

This application claims the benefit of U.S. provisional application No. 60/226,948, filed Aug. 22, 2000, U.S. provisional application No. 60/226,870, filed Aug. 22, 2000, and U.S. provisional application No. 60/226,871, filed Aug. 22, 2000, all of which are incorporated herein by reference.

### FIELD OF THE INVENTION

The field of the invention is improved specificity in treatment of diseases.

### BACKGROUND OF THE INVENTION

Liver diseases, and particularly viral hepatitis B and C remain a serious threat, and various treatment approaches have been developed. Depending on the drugs employed, the treatments may be classified as direct antiviral treatment, indirect antiviral treatment, or a combination of direct and indirect antiviral treatment.

Direct antiviral treatment interferes with virus replication and/or virus assembly. For example, nucleoside analogs can be employed to reduce viral replication by inhibiting the viral reverse transcriptase. However, nucleoside analogs are frequently associated with side effects, including anemia and/or neutropenia. Moreover, prolonged exposure to nucleoside analogs favors development of resistance to the drug in some virus strains. To circumvent at least some of the problems associated with drug resistance, cocktails of nucleoside analogs may be administered. Unfortunately, cocktails of nucleoside analogs typically only postpone the onset of drug resistance. Still further, nucleoside analogs generally do not exhibit selectivity between viral replication and replication in rapidly dividing cells of a host organism, thereby exhibiting significant cytotoxicity towards the host.

Alternatively, protease inhibitors may be employed to interrupt proper assembly of viral proteins. Protease inhibitors are highly specific towards viral proteases, thereby typically avoiding problems associated with limited selectivity between viral replication and replication in rapidly dividing host cells. However, even at relatively low dosages, side effects including nausea, diarrhea, diabetes, and kidney stones tend to occur. Moreover, some protease inhibitors are only poorly soluble in aqueous solution, thereby reducing the potential amount that can be delivered to a patient. Furthermore, viral resistance to some protease inhibitors has been shown to occur after prolonged treatment.

Indirect antiviral treatment may be used to stimulate the immune system to recognize a viral antigen, or to alter the cytokine balance of the immune system towards a Type 1 cytokine response, which is thought to aid the establishment of cellular immunity against virally infected cells. For example, IFN-alpha may be employed to treat chronic hepatitis C. However, many patients treated with IFN-alpha relapse after stopping treatment, and some of the patients undergo viral breakthrough during treatment. Furthermore, IFN-alpha tends to cause fever, headache, lethargy, loss of appetite, anxiety and depression, in lower dosages, and bone marrow suppression and low blood cell counts in higher dosages.

One or both of direct and indirect antiviral treatments can be achieved with co-administration of Ribavirin with Interferon-alpha, and has shown in many patients significant reduction of inflammation and serum ALT levels. Despite the relatively better therapeutic efficacy, various problems with Ribavirin still persist. For example, and especially at higher dosages, intracellular phosphorylation of Ribavirin in erythrocytes is known to contribute to hemolytic anemia. Moreover, phosphorylated Ribavirin tends to accumulate in erythrocytes, thereby reducing the effective dosage significantly. Consequently, relatively higher doses need to be administered to achieve a suitable dose of ribavirin in the hepatocytes.

Although there are various compositions and methods known in the art for targeted treatment of hepatic diseases, all or almost all of them suffer from one or more disadvantages. Therefore, there is still a need to provide improved compositions and methods for targeted treatment of diseases.

### SUMMARY OF THE INVENTION

The present invention is directed to methods and compositions for increasing selectivity of a drug. In general, a drug is covalently modified by a blocking group.

More particularly, in one aspect of the inventive subject matter, the blocking group is coupled to the drug via a nitrogen atom in the blocking group. The blocking group in the modified drug reduces metabolic conversion and sequestration (i.e., accumulation) of the drug in non-target cells, and the blocking group is enzymatically removed in the target cell.

In another aspect of the inventive subject matter, it is recognized that a metabolic conversion of a drug induces damage to a target cell, and it is further recognized that a blocking group attached to the drug via a nitrogen atom in the blocking group prevents metabolic conversion, and that the blocking group is cleaved from the drug in a target cell. Consequently, it is contemplated that modifying a drug with a blocking group, and administering the drug to a system comprising target and non-target cells reduce cytotoxicity.

In a further aspect of the inventive subject matter, it is recognized that a metabolic conversion of a drug in a non-target cell reduces the effective concentration of the drug, and it is further recognized that a blocking group attached to the drug via a nitrogen atom in the blocking group prevents metabolic conversion, and that the blocking is cleaved from the drug at a target cell. Consequently, it is contemplated that modifying a drug with a blocking group may reduce a dosage. It is also contemplated that the drug is administered to a system comprising target and non-target cells.

Contemplated drugs include a carboxamide group, and especially contemplated drugs are 1-beta-D-ribofuranosyl-1,2,4-triazole-3-carboxamide and 2-beta-D-ribofuranosyl-4-thiazolecarboxamide, which may also be in their respective L-isomeric form. While the blocking group is not restricted to a particular chemical nature, it is preferred that the blocking group comprises a nitrogen atom. An especially preferred blocking group is ═NH. Contemplated target cells are not limited to a particular cell type, and may or may not be infected with a virus, or they may be hyperproliferative. However, virus infected or hyperproliferative hepatocytes and neurons are especially preferred and non-target cells include erythrocytes.

Various objects, features, aspects and advantages of the present invention will become more apparent from the following detailed description of preferred embodiments of the invention, along with the accompanying drawing.

### BRIEF DESCRIPTION OF THE DRAWINGS

FIGS. 1A and 1B are schematic illustrations of uptake and retention of an exemplary drug according to the inventive subject matter.

FIG. 2 is an exemplary synthetic scheme for the synthesis of Ribavirin.

FIG. 3 is an exemplary synthetic scheme for the synthesis of modified Ribavirin.

### DETAILED DESCRIPTION

As used herein, the term “pharmacological effect” refers to any alteration of metabolism, replication, structure, or life span of a cell in a cell containing system, which is caused by a molecule that is added to the system. For example, inhibition of an anabolic, catabolic, or polymerase-type reaction catalyzed by an enzyme is considered a pharmacological effect. Similarly, depolymerization of tubulin by KinI kinesins is considered a pharmacological effect under the scope of this definition. In contrast, allosteric inhibition of an enzyme by a metabolite produced within the cell in a system is not regarded a pharmacological effect, because the allosteric inhibitor was not added to the system.

As further used herein, the term “target cell” refers to a cell in which a drug is intended to exhibit a pharmacological effect. For example, a virus-infected hepatocyte is considered a target cell for the drug Ribavirin. In contrast, the term “non-target cell” encompasses all cells in a cell-containing system that are not target cells.

It is generally known that while certain drugs are intended to exhibit their pharmacological effect on particular cells (i.e., target cells), non-target cells may metabolize those drugs at a significant rate, frequently leading to undesirable non-specific side effects. The inventors have discovered that such undesired metabolic conversion at (i.e., in, or on the surface of) non-target cells can be prevented by modifying a drug with a blocking group that is selectively removed in a target cell, thereby increasing the selectivity of a pharmacological effect of a drug, while concomitantly reducing the cytotoxicity and the dosage of the drug.

In one aspect of the inventive subject matter, the selectivity of the pharmacological effect of a drug is increased by a method in which in one step a drug is identified as having a desired pharmacological effect on a target cell. In a further step, the drug is modified with a blocking group, wherein the blocking group is covalently attached to the drug via a nitrogen atom in the blocking group. The blocking group further reduces accumulation of the drug in a non-target cell, and is enzymatically removed from the drug in the target cell. As used herein, the term “selectivity” of a drug with respect to a pharmacological effect refers to the propensity of a drug to preferentially exert the pharmacological effect on a target cell towards a treatment goal as opposed to exerting the pharmacological effect on a non-target cell towards an undesired side effect of the drug in the non-target cell.

For example, the selectivity of the pharmacological effect of 1-beta-D-ribofuranosyl-1,2,4-triazole-3-carboxamide (Ribavirin, Structure 1) is increased by covalently coupling an ═NH group to Ribavirin to form a carboxamidine group. Ribavirin is known to have antiviral properties in hepatitis virus-infected hepatocytes (e.g., see review Marcellin, P. and Benhamou J.; Treatment of chronic viral hepatitis, Baillieres Clin Gastroenteol 1994 June; 8(2):233-53). It is also known, that Ribavirin is readily phosphorylated in erythrocytes to the pharmacologically active form Ribavirin-phosphate at a significant rate (e.g., Homma, M. et al. High-performance liquid chromatographic determination of Ribavirin in whole blood to assess disposition in erythrocytes; Antimicrob Agents Chemother 1999 November; 43(11):2716-9), which reduces the selective pharmacological effect. Surprisingly, the inventors have discovered that Ribavirin modified with a ═NH group (1-beta-D-ribofuranosyl-1,2,4-triazole-3-carboxamidine, Structure 2) is not, or is only insignificantly phosphorylated in erythrocytes, and that the ═NH group in the 1-beta-D-ribofuranosyl-1,2,4-triazole-3-carboxamidine is specifically and enzymatically removed in hepatocytes to form 1-beta-D-ribofuranosyl-1,2,4-triazole-3-carboxamide.

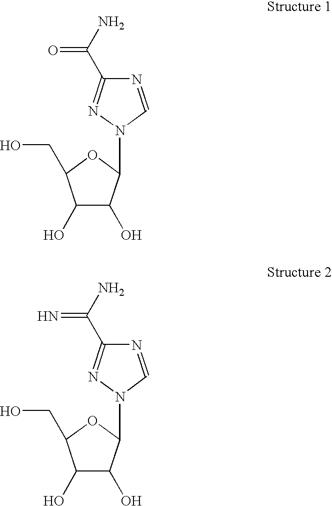

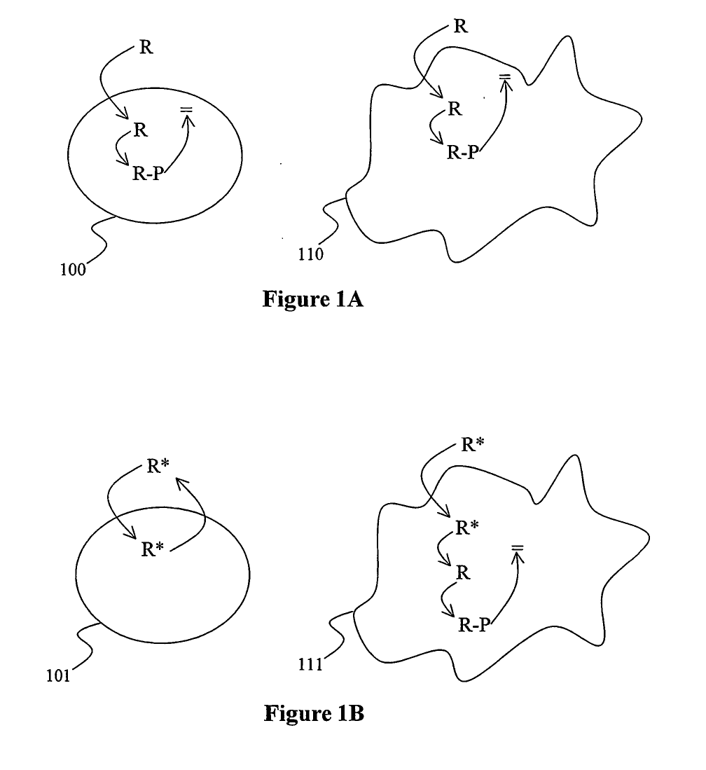

The reduction in selective accumulation of modified Ribavirin is illustrated in FIGS. 1A and 1B. In FIG. 1A, an erythrocyte (non-target cell) 100 is presented with Ribavirin (R). Ribavirin enters the erythrocyte, and is phosphorylated to the pharmacologically active Ribavirin-phosphate (R-P), which is retained in the erythrocyte. Similarly, a hepatocyte (target cell) 110 is presented with Ribavirin (R). Ribavirin enters the hepatocyte, and is phosphorylated to the pharmacologically active Ribavirin-phosphate (R-P), which is retained in the hepatocyte. In FIG. 1B, an erythrocyte (non-target cell) 101 is presented with modified Ribavirin (R*, 1-beta-D-ribofuranosyl-1,2,4-triazole-3-carboxamidine). The modified Ribavirin enters the erythrocyte, however, it is not phosphorylated and can therefore exit the erythrocyte. Similarly, a hepatocyte (target cell) 111 is presented with modified Ribavirin (R*). The modified Ribavirin enters the hepatocyte, and is enzymatically deaminated to Ribavirin, which is subsequently phosphorylated to the pharmacologically active phosphorylated Ribavirin (R-P), which is retained in the hepatocyte.

In an alternative aspect of the inventive subject matter, it is contemplated that there are many drugs other than Ribavirin suitable for the inventive concept presented herein. Generally, appropriate drugs include drugs that are metabolized, activated and/or inactivated in a cell other than a target cell, and particularly contemplated drugs include nucleosides, nucleotides, nucleoside analogs and nucleotide analogs. For example, Tiazofurin (2-beta-D-ribofuranosyl-4-thiazolecarboxamide) is a nucleoside analog with a carboxamide group that can readily be modified with an ═NH group to yield the corresponding carboxamidine. In another example, an alternative drug comprises the nucleoside uracil, or the nucleoside analog 5′-fluoro uracil (5′-FU).

In still further alternative aspects of the inventive subject matter, the blocking group need not be limited to a ═NH group, but may also include various primary and secondary amines, so long as the blocking group can be covalently coupled to the drug via the nitrogen atom. The term “blocking group” as used herein refers to a chemical group that can be covalently attached to a drug, and when attached to the drug, blocks at least one metabolic conversion of the drug. As also used herein, the term “metabolic conversion” of a drug refers to any intra and/or extracellular chemical change of a drug that is brought about by the metabolism of a cell or cellular system, and particularly includes enzymatic degradation (e.g., oxidation, hydrolytic cleavage) and enzymatic modification (e.g., glycosylation, phosphorylation).

It is generally contemplated that suitable blocking groups have the structure—N(R₁)(R₂) or ═NR₁, wherein R₁ and R₂ are independently hydrogen, linear or branched alkyl, alkenyl, alkynyl, aralkyl, aralkenyl, or aralkynyl, aryl, all of which may further comprise heteroatoms including nitrogen, oxygen, sulfur, or a halogen. It is especially preferred, however, that alternative blocking groups are enzymatically removable from the drug, and particularly contemplated enzymes include liver specific aminohydrolases, including deaminases (e.g., adenosine or cytosine deaminase), liver deamidases (e.g., nicotinamide deamidase) and liver transaminases (glutamate-pyruvate transaminase).

Contemplated blocking groups may be covalently bound to various positions of the drug molecule, and while it is generally preferred that contemplated drugs are modified on a carboxamide moiety, various positions other than a carboxamide group are also contemplated, especially carbonyl groups (e.g., a carboxylic acid, and a keton-type carbonyl). For example, each of the carbonyl groups in the ring portion of uracil or its analog 5′-FU may be modified by a blocking group.

Although not limiting to the inventive concept presented herein, it is contemplated that the blocking group may inactivate the drug, or prevent subsequent activation once the modified drug is presented to a non-target cell. For example, where the blocking group is coupled to the drug at a position that is essential for specific interaction of the drug with a target molecule (e.g., a receptor or substrate binding site), the blocking group may inactivate the drug. On the other hand, the blocking group may be coupled to the drug at a position that may prevent metabolic activation.

Depending on the chemical nature of the drug and/or blocking group, it is contemplated that the blocking group may replace a functional group or substituent, or that the blocking group is attached to a functional group or substituent. For example, where the drug is Ribavirin and the blocking group is an ═NH group, the oxygen atom in the carboxamide group of Ribavirin is replaced by the ═NH group. On the other hand, where the drug comprises a nucleophilic group (e.g., —O⁻), and the blocking group comprises a secondary amine with a suitable leaving group, the secondary amine may be attached to the nucleophilic group.

With respect to the step of modifying the drug, it is contemplated that the modification may comprise an organo-synthetic modification, an enzymatic modification, or a de-novo synthesis to produce the modified drug. For example, where the drug comprises an activated carbonyl function, amidation of the carbonyl atom may be achieved in a single nucleophilic exchange reaction. Alternatively, and especially where the drug has numerous reactive groups other than the group to which the blocking group is to be attached, a de-novo synthesis of the modified drug may be economically more attractive. It is especially contemplated that appropriate drugs may also be enzymatically modified by introducing the blocking group into the drug in a reaction that employs the drug and the blocking group as enzymatic substrates. Where available, it is preferred that the enzyme for such modifications is derived from the target cell (e.g., from an allogenic or xenogenic source, or from a recombinant source expressing the gene coding for the enzyme).

It should be appreciated that depending on the type of the non-target cell, and depending on the chemical nature of the drug and/or blocking group, the prevention of accumulation of the drug in the non-target cell may be achieved by at least one of various mechanisms, including reduction of uptake through drug-specific transporters, reduction of metabolic conversion into forms that will be retained (e.g., due to additional or new electrical charge, change in hydrophobicity, or change in recognition by an exporter), or, due to increased export from the non-target cell (e.g., due to an secretory signal in the blocking group) prevention of accumulation of the drug may be achieved. For example, it is contemplated that where the drug is a nucleoside analog, and the non-target cell has a nucleoside transporter that selectively imports nucleosides without a lipophilic moiety; adding a lipophilic moiety as the blocking group to the drug may prevent accumulation of the drug. In another example, it is contemplated that phosphorylation (and concomitant accumulation) of various nucleosides in erythrocytes can be prevented by converting a carboxamide group into a carboxamidine group (supra).

With respect to the enzymatic removal of the blocking group in the target cell it is contemplated, that depending on the type of the target cell, the blocking group and the drug, the enzymatic removal may vary considerably. Enzymatic removal may include enzymes from various classes, including hydrolases, transferases, lyases, and oxidoreductases, and particularly preferred subclasses are adenosine and cytosine deaminases, arginases, transaminases, and arylamidases. It should further be appreciated that contemplated enzymes for the enzymatic removal of the blocking group may exclusively be expressed in the target cells, however, in alternative aspects of the inventive subject matter appropriate enzymes may also be expressed in cells other than the target cells, so long as the enzyme is not ubiquitously expressed in all cells in a cell containing system. It should further be appreciated that contemplated enzymes are natively expressed (i.e., are non-recombinant) in the respective target cells under normal and/or pathological conditions. For example, it is known that glutamine-pyruvate transaminase is constitutively expressed with relatively high selectivity in liver cells, and may therefore be a suitable enzyme for removal of a blocking group. Alternatively, it is known that cytosine deaminase is expressed in relatively high quantities in colon cancer cells, but not, or only in minor quantities in normal colon cells.

In another aspect of the inventive subject matter, the cytotoxicity of a drug to a non-target cell is reduced by a method in which in one step it is recognized that a metabolic conversion of a drug in a non-target cell induces damage to the non-target cell. The term “cytotoxicity” as used herein refers to an undesired pharmacological effect on a non-target cell, wherein the undesired pharmacological effect particularly includes inhibition of replication, energy-metabolism, or includes cell death. In a further step, the drug is modified with a blocking group, wherein the blocking group is covalently coupled to the drug via a nitrogen atom in the blocking group, and wherein the blocking group reduces the metabolic conversion of the drug in the non-target cell, and is enzymatically cleaved from the drug in the target cell. In yet a further step, the drug is administered to a system comprising the target cell and the non-target cell, wherein the blocking group is covalently coupled to the drug.

In a preferred aspect of reducing the cytotoxicity of a drug to a non-target cell, the metabolic conversion includes phosphorylation of the drug to the corresponding drug-phosphate in an erythrocyte. For example, it is well known in the art that phosphorylation of the antiviral drug Ribavirin in various cells produces pharmacologically active Ribavirin-5′-monophosphate (e.g., Homma, M. et al. High-performance liquid chromatographic determination of Ribavirin in whole blood to assess disposition in erythrocytes; Antimicrob Agents Chemother 1999 November; 43(11): 2716-9), a compound involved in the inhibition of inosine monophosphate dehydrogenase (IMPDH). Unfortunately, Ribavirin-5′-monophosphate has a pronounced cytotoxic effect on erythrocytes (De Franceschi, et al.; Hemolytic anemia induced by ribavirin therapy in patients with chronic hepatitis C virus infection: role of membrane oxidative damage, Hepatology 2000 April; 31(4): 997-1004), and it is consequently recognized that prevention or reduction of formation of Ribavirin-5′-monophosphate in erythrocytes will significantly reduce the cytotoxicity of Ribavirn.

It should be appreciated, however, that various metabolic conversions of a drug in a non-target cell other than phosphorylation are also contemplated, including oxidation, reduction, hydrolytic cleavage of a covalent bond within the drug, addition or removal of pendent groups, and ring-opening reactions. For example, it is contemplated that where the non-target cell is a hepatocyte, the metabolic conversion may include various enzymatic detoxification or solubilization reactions known to occur in the liver (e.g., glycosylation, cytochrome P₄₅₀-mediated oxidation, etc.). In another example, metabolic conversions may include phosphatase or esterase activity.

Depending on the type of metabolic conversion, the conversion may be limited to a single type of non-target cell, but may also occur in more than one cell type. For example, where the non-target cell has a relatively high rate of nucleic acid synthesis, and the metabolic conversion is mediated by an enzyme involved in the nucleic acid synthesis, various types of fast growing cells may exhibit metabolic conversion. On the other hand, metabolic conversion may also be locally limited through accessibility of the drug to a particular set of cells or organs.

It should be appreciated that various well known laboratory procedures may be employed to recognize and/or verify that a blocking group covalently coupled to the drug reduces metabolic conversion in the non-target cell. For example, where the non-target cells are cultured in vitro, it is contemplated that the non-target cells may be incubated with the corresponding radiolabeled drug, and that the metabolites of the radiolabeled drugs may then be identified by various assays, including immunoassays, thin layer chromatography, or GC-MS. Alternatively, where the non-target cells are located in a mammal, a tissue biopsy may provide a sufficient specimen to isolate and identify metabolites of the administered drug.

It is further contemplated that the type of damage to the non-target cell may vary substantially, and may range from slowing the cellular metabolism in the non-target cell-to-cell death. For example, where the metabolic conversion produces an inhibitor of an enzyme located in the glycolytic pathway, energy for the non-target cell may be provided at least in part through salvage pathways. Likewise, where the metabolic conversion of a drug into an enzyme inhibitor proceeds at a relatively slow rate, up-regulation of expression of the enzyme affected by the inhibitor may almost completely compensate for the reduction in number of active sites. On the other hand, where the metabolic conversion produces a radical species, lipid peroxidation may result in severe membrane damage and subsequent death of the cell.

It is further contemplated that the damage resulting from the metabolic conversion of the drug may be caused directly or indirectly. For example, where the metabolic conversion produces an enzyme inhibitor blocking an enzyme, the damage is considered direct. On the other hand, where the metabolic conversion produces an intermediate, which after subsequent intracellular or exatracellar modification is further converted to an enzyme inhibitor, the damage is considered indirect.

With respect to the step of administering the drug to a system, it is contemplated that suitable drugs will be administered in any appropriate pharmaceutical formulation, and under any appropriate protocol. Thus, administration may take place orally, parenterally (including subcutaneous injections, intravenous, intramuscularly, by intrasternal injection or infusion techniques), by inhalation spray, rectally, topically and so forth, and in dosage unit formulations containing conventional non-toxic pharmaceutically acceptable carriers, adjuvants and vehicles. For example, it is contemplated that appropriate drugs can be administered orally as pharmacologically acceptable salts, or alternatively intravenously in physiological saline solution (e.g., buffered to a pH of about 7.2 to 7.5). Conventional buffers such as phosphates, bicarbonates or citrates can be used for this purpose. Furthermore, it is contemplated that it is well within the ordinary skill of the art to modify the route of administration and dosage regimen of a particular drug in order to manage the pharmacokinetics of the present compounds for maximum beneficial effect in patients.

In certain pharmaceutical administration forms, pro-drug forms of the drugs are contemplated. One of ordinary skill in the art will recognize how to readily modify contemplated drugs to pro-drug forms to facilitate delivery of active compounds to a target site within the host organism or patient. One of ordinary skill in the art will also take advantage of favorable pharmacokinetic parameters of the pro-drug forms, where applicable, in delivering the present compounds to a targeted site within the host organism or patient to maximize the intended effect of the compound.

It should further be appreciated that contemplated drugs may be administered alone or in combination with other pharmacologically active agents, which may be administered separately or together and when administered separately, administration may occur simultaneously or separately in any order. Contemplated pharmacologically active agents include anti-viral agents such as interferon (e.g., interferon α and γ), anti-fungal agents such as tolnaftate, Fungizone™, Lotrimin™, Mycelex™, Nystatin and Amphoteracin; anti-parasitics such as Mintezol™, Niclocide™, Vermox™, and Flagyl™; bowel agents such as Immodium™, Lomotil™ and Phazyme™; anti-tumor agents such as interferon α and γ, Adriamycin™, Cytoxan™, Imuran™, Methotrexate, Mithracin™, Tiazofurin™, Taxol™; dermatologic agents such as Aclovate™, Cyclocort™, Denorex™, Florone™, Oxsoralen™, coal tar and salicylic acid; migraine preparations such as ergotamine compounds; steroids and immunosuppresants not listed above, including cyclosporins, Diprosone™, hydrocortisone; Floron™, Lidex™, Topicort and Valisone; and metabolic agents such as insulin, and other drugs which may not fit into the above categories, including cytokines such as IL2, IL4, IL6, IL8, IL10, and IL12.

With respect to the dosage of contemplated drugs and pharmacologically active agents, it is contemplated that a therapeutically effective amount will vary with the condition to be treated, its severity, the treatment regimen to be employed, the pharmacokinetics of the agent used and the patient (animal or human) being treated. It is further contemplated that various dosages are appropriate, including dosages between 0.5 mg/kg and 0.1 mg/kg and less, but also dosages between 0.5 and 1.0 mg/kg and more. While it is generally preferred that the system comprising the target cell and the non-target cell is a mammal (most preferably a human), various alternative systems are also appropriate, and particularly include in vitro cell and tissue culture.

With respect to the drug, the blocking group, the step of modifying the drug, the target cell and the non-target cell in contemplated methods of reduction of cytotoxicity of a drug to a non-target cell, the same considerations as described above apply.

In still a further aspect of the inventive subject matter, the dosage of a drug in a system is reduced by a method in which a drug is provided, wherein metabolic conversion of the drug in a non-target cell reduces the concentration of the drug in a system comprising the non-target cell and a target cell. In a further step, the drug is modified with a blocking group, wherein the blocking group is covalently coupled to the drug via a nitrogen atom in the blocking group, and wherein the blocking group reduces the metabolic conversion of the drug in the non-target cell. In a subsequent step, the drug is administered to the system, wherein the blocking group is covalently coupled to the drug, and wherein the blocking group is enzymatically removed from the drug in the target cell.

In a preferred aspect of reducing the dosage of a drug in a system, the drug is Ribavirin, the target cell is a hepatocyte infected with a virus, and the non-target cell is an erythrocyte. It is well known in the art (supra), that Ribavirin is metabolically converted to Ribavirin-phosphate and that Ribavirin-phosphate is retained in the erythrocytes, thereby significantly lowering the concentration of Ribavirin. Ribavirin is modified by covalently attaching a ═NH blocking group to the carboxamide carbon, thereby replacing the carbonyl oxygen in the carboxamide. It has been shown (infra) that metabolic conversion of Ribavirin modified with a ═NH blocking group is significantly reduced in erythrocytes. It is further contemplated that the preferred administration of modified Ribavirin is in a single oral dose of 50 mg-300 mg in a human.

Ribavirin is known to be orally administered to a human as an antiviral drug in at least one single dose of about 600 mg-1200 mg. The initial concentration of Ribavirin in the system (e.g., a human) is between about 1 μM and several hundred μM, however, the Ribavirin concentration is typically reduced in the system by sequestration into erythrocytes within 24 hours to about 85% to 50% of the initial concentration due to phosphorylation of Ribavirin in the erythrocytes. The inventors have shown, that modification of Ribavirin to 1-beta-D-ribofuranosyl-1,2,4-triazole-3-carboxamidine significantly reduces the amount of phosphorylation (infra) of Ribavirin. Therefore, it is contemplated that all or almost all of the initial concentration of Ribavirin is available for the desired pharmacological effect in the target cells. Consequently, it is contemplated that modification of Ribavirin with a blocking group can be employed to reduce the dosage of Ribavirin by about 5 wt %, preferably by about 10 wt %, more preferably by 25 wt % and most preferably by 50 wt %.

It should be appreciated, however, that various dosages other than 600 mg-1200 mg are also contemplated, including dosages of 200 mg-600 mg, dosages of 20 mg-200 mg, and less. For example, where Ribavirin is employed as an immunomodulatory drug, lower dosages of about 100 mg-300 mg may be sufficient. On the other hand, where relatively high concentrations of the drug are desired, dosages of 600 mg-1800 mg, and more, are contemplated. It should also be appreciated that depending on the particular metabolic conversion the reduction of the dosage may vary considerably. For example, where the metabolic conversion is relatively rapid and takes place in a plurality of non-target cells, reductions of the dosage of between 25 wt % and 80 wt %, and more, are contemplated. On the other hand, where the metabolic conversion is relatively slow, reductions of the dosage of between 25 wt % and 5 wt %, and less, are contemplated.

With respect to the drug, the blocking group, the metabolic conversion, the step of modifying the drug, the system, the step of administering the drug, the target cell and the non-target cell in contemplated methods of reducing the dosage of a drug, the same considerations as described above apply.

### EXAMPLES

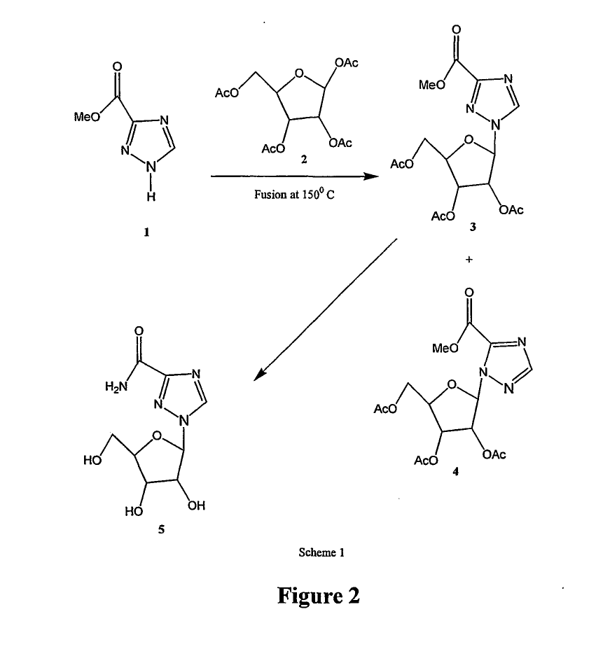

(a) An exemplary synthesis of Ribavirin is depicted in FIG. 2, which follows a procedure as outlined below.

Methyl 1-(2,3,5-tri-O-acetyl-β-D-ribofuranosyl)-1,2,4-triazole-3-carboxylate (3) and

Methyl 1-(2,3,5-tri-O-acetyl-β-D-ribofuranosyl)-1,2,4-triazole-5-carboxylate (4)

A mixture of methyl-1,2,4-triazole-3-carboxylate (25.4 g, 200 mmol) (1), 1,2,3,5-tetra-O-acetyl-β-D-ribofuranose (63.66 g, 200 mmol) (2) and bis(p-nitrophenyl)phosphate (1 g) were placed in a RB flask (500 mL). The flask was placed in a pre-heated oil bath at 165-175° C. under water aspirator vacuum with stirring for 25 min. The acetic acid displaced was collected in an ice-cold trap that is placed between aspirator and the RB flask. The flask was removed from the oil bath and allowed to cool. When the temperature of the flask reached roughly to 60-70° C., EtOAc (300 mL) and sat. NaHCO₃ (150 mL) were introduced, and extracted in EtOAc. The aqueous layer was extracted again with EtOAc (200 mL). The combined EtOAc extract was washed with sat. NaHCO₃ (300 mL), water (200 mL) and brine (150 mL). The organic extract was dried over anhydrous Na₂SO₄, filtered and the filtrate evaporated to dryness. The residue was dissolved in EtOH (100 mL) and diluted with MeOH (60 mL) which on cooling at 0° C. for 12 h provided colorless crystals. The solid was filtered, washed with minimum cold EtOH (20 mL) and dried at high vacuum over solid NaOH to give 60 g (78%). The filtrate was evaporated to dryness and purified on silica column using CHCl₃→EtOAc (9:1) as the eluent. Two products were isolated from the filtrate, a fast moving product of about 8.5 g (11%), and a slow moving product of about 5 g (6.5%). The slow moving product matched with crystallized product. The fast moving product was found to be 4 and obtained as foam. The combined yield of the 3 was 65 g (84%); mp 108-110° C.; ¹H NMR (CDCl₃) of 3: δ2.11 (s, 3H, COCH₃), 2.12 (s, 3H, COCH₃), 2.13 (s, 3H, OCH₃), 3.99 (s, 3H, COCH₃), 4.22 (dd, 1H), 4.46 (m, 2H), 5.55 (t, 1H, J=6.0 Hz), 5.75 (m, 1H), 6.05 (d, 1H, C_{1′}H J=3.6 Hz) and 8.41 (s, 1H, C₅H). Anal. (C₁₅H₁₉N₃O₉) C, H, N. ¹H NMR (CDCl₃) of 4: δ2.02 (s, 3H, COCH₃), 2.10 (s, 3H, COCH₃), 2.12 (s, 3H, OCH₃), 4.00 (s, 3H, COCH₃), 4.14 (m, 1H), 4.42 (m, 2H, 5.76 (t, 1H), 5.81 (m, 1H), 6.94 (d, 1H, C_{1′}H J=2.1 Hz), 8.03 (s, 1H, C₅H). Anal. (C₁₅H₁₉N₃O₉) C, H, N.

1-β-D-Ribofuranosyl-1,2,4-triazole-3-carboxamide (5)

Methyl-1-(2,3,5-tri-O-acetyl-β-D-ribofuranosyl)-1,2,4-triazole-3-carboxylate (62 g, 161 mmol) (3) was placed in a steel bomb and treated with freshly prepared methanolic ammonia (350 mL, prepared by passing dry ammonia gas into dry methanol at 0° C. until saturation) at 0° C. The steel bomb was closed and stirred at room temperature for 18 h. The steel bomb was cooled to 0° C., opened and the content evaporated to dryness. The residue was treated with dry EtOH (100 mL) and evaporated to dryness. The residue obtained was triturated with acetone to give a solid, which was filtered and washed with acetone. The solid was dried overnight at room temperature and dissolved in hot EtOH (600 mL) and water (10 mL) mixture. The volume of the EtOH solution was reduced to 150 mL by heating and stirring on a hot plate. The hot EtOH solution on cooling provided colorless crystals, which was filtered, washed with acetone and dried under vacuum. Further concentration of the filtrate gave additional material. Total yield: 35 g (89%); mp 177-179° C.; [α]²⁰_{D}-35.3 (c, 10, H₂O); ¹H NMR (Me₂SO-d₆): δ3.46 (m, 1H, C_{5′}H), 3.60 (m, 1H, C_{5′}H), 3.94 (m, 1H), C_{4′}H), 4.12 (m, 1H) 4.34 (m, 1H), 4.95 (t, 1H, C_{5′}OH), 5.22 (d, 1H), 5.60 (d, 1H), 5.80 (d, 1H, J=3.9 Hz, C_{1′}H), 7.64 (bs, 1H, NH₂), 7.84 (bs, 1H, NH₂), 8.87 (s, 1H, C₅H). ¹³C NMR (Me₂SO-d₆) δ61.8, 70.2, 74.4, 86.0, 91.6, 144.9, 157.4, 160.6. Anal. (C₈H₁₂N₄O₅) C, H, N.

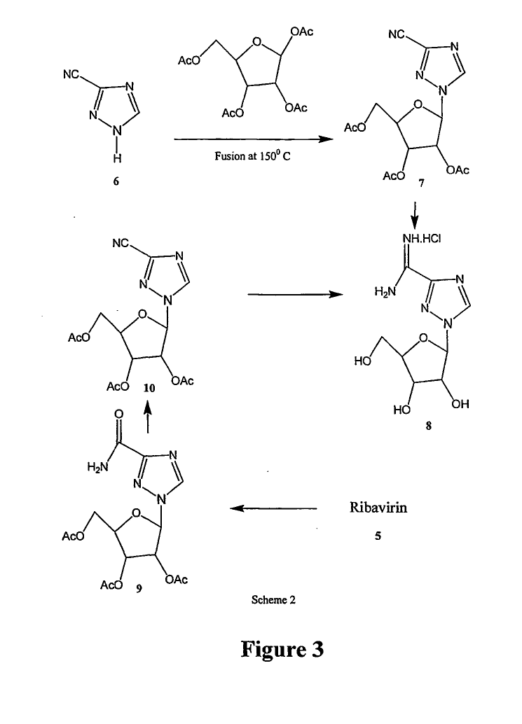

(b) An exemplary synthesis of Ribavirin modified with a ═NH group is depicted in FIG. 3, which follows a procedure as outlined below.

3-Cyano-1-(2,3,5-tri-O-acetyl-β-D-ribofuranosyl)-1,2,4-triazole (7)

A mixture of 3-cyano-1,2,4-triazole (18.8 g, 200 mmol) (6), 1,2,3,5-tetra-O-acetyl-β-D-ribofuranose (63.66 g, 200 mmol) and bis(p-nitrophenyl)phosphate (1 g) was placed in a RB flask (500 mL). The flask was placed in a pre-heated oil bath at 165-175° C. under water aspirator vacuum with stirring for 25 minutes. The acetic acid displaced was collected in an ice-cold trap that is placed between aspirator and the RB flask. The flask was removed from the oil bath and allowed to cool. When the temperature of the flask reached roughly to 60-70° C., EtOAc (300 mL) and sat. NaHCO₃ (150 mL) were introduced, and extracted in EtOAc. The aqueous layer was extracted again with EtOAc (200 mL). The combined EtOAc extract was washed with sat. NaHCO₃ (300 mL), water (200 mL) and brine (150 mL). The organic extract was dried over anhydrous Na₂SO₄, filtered and the filtrate evaporated to dryness. The residue was dissolved in ether (100 mL) which on cooling at 0° C. for 12 h provided colorless crystals. The solid was filtered, washed with minimum cold EtOH (20 mL) and dried at high vacuum over solid NaOH. Yield: 56.4 g (80%). mp 96-97° C. ¹HMR (CDCl₃): δ2.11 (s, 3H, COCH₃), 2.13 (s, 3H, COCH₃), 2.14 (s, 3H, COCH₃), 4.22 (dd, 1H), 4.46 (m, 2H), 5.52 (t, 1H, J=6.0 Hz), 5.70 (m, 1H), 6.01 (d, 1H, C_{1′}H J=3.6 Hz) and 8.39 (s, 1H, C₅H). Anal. Calc. For C₁₄H₁₆N₄O₇ (352.30): C, 47.73; H, 4.58; N, 15.90. Found: C, 47.70; H, 4.63; N, 16.01.

1-β-D-Ribofuranosyl-1,2,4-triazole-3-carboxamidine Hydrochloride (8)

A mixture of 7 (14.08 g, 40.0 mmol), NH₄Cl (2.14 g, 40.0 mmol) and anhydrous ammonia (150 ml) was heated in a steel bomb at 85° C. for 18 h. The steel bomb was cooled, opened and the contents were evaporated to dryness. The residue was crystallized from MeCN-EtOH to provide 10.6 g (95%) of 8. Mp 177-179° C. ¹HMR(DMSO-d₆): δ3.44-4.2 (m, 3H), 4.40 (m, 2H), 5.04 (t, 1H), 5.29 (m, 1H), 5.74 (m, 1H), 5.87 (d, 1H, C_{1′}H), 8.96 (bs, 3H) and 9.17 (s, 1H, C₅H). Anal. Calc. For C₈H₁₄ClN₅O₄ (279.68): C, 34.35; H, 5.05; N, 25.04; Cl, 12.69. Found: C, 34.39; H, 5.10; N, 25.14; Cl, 12.71.

An exemplary alternative route starting from Ribavirin proceeds as follows:

2′,3′, 5′-Tri-O-acetyl-1-β-D-ribofuranosyl-1,2,4-triazole-3-carboxamide (9)

A suspension of 1-β-D-ribofuranosyl-1,2,4-triazole-3-carboxamide (28.4 g, 116.4 mmol) (Ribavirin) in acetic anhydride (200 mL) and pyridine (50 mL) was stirred at room temperature overnight. The resulting clear solution was concentrated in vacuo to yield a clear foam (43.1 g, quantitive). This foam was homogenous on TLC and used directly for the next step without purification. A small amount was purified by flash chromatography to yield an analytical sample; ¹H NMR (300 MHz), DMSO-d₆) δ2.01, 2.08, 2.09 (3s, 9 H, COCH₃), 4.10 (m, 1 H), 3.52 (m, 2 H), 5.58 (t, 1 H), 5.66 (m, 1 H); 6.33 (d,.1 H, J=3.0 Hz, C₁H), 7.73, 7.92, (2 s, 2 H, CONH₂), 8.86 (s, 1 H, C₅H triazole). Anal. (C₁₀H₁₈N₄O₈) C, H,N.

3-Cyano-2′,3′,5′-tri-O-acetyl-1-β-D-ribofuranosyl-1,2,4-triazole (10)

To a solution of 9 (43.1 g, 116.4 mmol) in chloroform (500 mL) was added triethylamine (244 mL) and the mixture cooled to 0° C. in an ice-salt bath. Phosphorus oxychloride (30.7 mL, 330 mmol) was added drop wise with stirring and the solution allowed to warn to room temperature. After the mixture was stirred at room temperature for 1 h, TLC (hexane/acetone 3:1) indicated complete disappearance of starting material. The brown reaction mixture was concentrated to dryness in vacuo and the residue dissolved in chloroform (500 mL). This organic solution was washed with saturated aqueous sodium bicarbonate (3×200 mL), dried over anhydrous sodium sulfate, and concentrated in vacuo. The residue was chromatographed over silica gel (flash chromatography) with 20% acetone in hexane to yield 33.14 g (81% from ribavirin) of pure 10 as an amorphous solid. This solid was identical in all respects with an authentic sample: mp 101-103° C.; IR (potassium bromide) ν 2250 (CN), 1750 (C═O), cm⁻¹; ¹H NMR (300 MHz, CDCl₃) δ2.04, 2.06, 2.07 (3 s, 9 H, acetyl methyls), 4.15 (dd, 1 H), 4.40 (m, 1 H), 5.47 (t, 1 H), 5.63 (dd, 1 H), 5.95 (d, 1 H, J=3.2 Hz, C₁H), 8.34 (s, 1 H, C₅H triazole).

1-β-D-Ribofuranosyl-1,2,4-triazole-3-carboxamidine Hydrochloride (8)

To a suspension of 10 (4.0 g, 11.4 mmol) in methanol (100 mL) was added a molar solution of methanolic sodium methoxide (12 mL) and the mixture stirred at room temperature overnight. The solution was acidified to pH 4 with methanol washed Dowex H+ resin, the resin was filtered, and the filtrate was concentrated to dryness in vacuo. The residue was dissolved in a minimum amount of methanol (15 mL) and transferred to a pressure bottle. Ammonium chloride (0.61 g, 11.4 mmol) and a solution of methanol saturated at 0° C. with dry ammonia gas (75 mL) were added, the bottle was sealed, and the solution was stirred at room temperature overnight. The solution was concentrated to dryness in vacuo and the resulting residue crystallized from acetonitrile/ethanol to yield 8 as a crystalline solid (2.95 g, 93%). This sample was identical in all respects with an authentic sample.

In yet another alternative route, 1-β-D-Ribofuranosyl-1,2,4-triazole-3-carboxamidine Hydrochloride (8) can be produced by an enzymatic reaction using a culture of a microorganism, intact cells of a microorganism, or a cell extract as an enzyme source (under non-proliferating conditions of the microorganism). 3-Cyano-1-(2,3,5-tri-O-acetyl-β-D-ribofuranosyl)-1,2,4-triazole (7) can be produced by causing 3-cyano-1,2,4-triazole or salt thereof and a ribose donor to contact in the presence of an enzyme source based on the microorganism. Then, compound 7 will be transformed into (8) by treating (7) with liquid ammonia solution. Alternatively, 1,2,4-triazole-3-carboamidine hydrochloride can react with ribose donor in the presence of an enzyme to produce directly (8).

(c) Deamination of Modified Ribavirin to Ribavirin in the Liver

In mice, after repeated oral dosing of ³H-Ribavirin and ³H-(═NH) modified Ribavirin at a dose of 300 mg/kg daily for 8 days, the medium, minimum, radioactivity concentration Cₘᵢₙ in the liver was significantly lower for Ribavirin as compared to modified Ribavirin. It should be especially pointed out, that Ribavirin accounted for about 90% and ribofuranosyl triazole carboxylic acid (RTCA) accounted for about 10% of liver radioactivity in mice treated with Ribavirin. In contrast, modified Ribavirin accounted for about 30% and Ribavirin accounted for about 70% of liver radioactivity in mice treated with modified Ribavirin (see also Table 1).

TABLE 1
Liver Radioactivity in Mice

| | | ³H-(=NH) |
|---|---|---|
| | ³H-Ribavirin | modified Ribavirin |
| Total Liver Radioactivity | 18.4 μg equiv/g | 23.8 μg equiv/g |
| RTCA | ˜1.8 μg equiv/g | not detected |
| Ribavirin | ˜16.6 μg equiv/g | ˜16.6 μg equiv/g |
| Modified Ribavirin | not detected | ˜7.2 μg eguiv/g |

(d) Differential Radioactivity Distribution of Ribavirin and (═NH) Modified Ribavirin in Red Blood Cells (RBC)

Ribavirin has been shown to be phosphorylated in RBCs, and it has further been suggested that phosphorylated Ribavirin is a causative agent in hemolytic anemia observed in long-term treatment or high dosages of Ribavirin in humans. Remarkably, modified (═NH)-modified Ribavirin is not directly transported into RBCs as evidenced by in vitro studies (data not shown), and it is consequently contemplated that modified Ribavirin will accumulate in RBCs only after deamination in the liver to Ribavirin and subsequent phosphorylation into the corresponding phosphates as shown in Table 2 below.

In mice, after repeated oral dosing of ³H-Ribavirin and ³H-(═NH) modified Ribavirin at a dose of 300 mg/kg daily for 8 days, the medium, minimum, radioactivity concentration Cₘᵢₙ in RBCs was significantly lower for modified Ribavirin than Ribavirin. As judged from the differential data shown in Tables 1 and 2, the therapeutic index (i.e., the ratio between liver Ribavirin concentration and RBC Ribavirin concentration) for modified Ribavirin is about three times of that of Ribavirin.

In portal, vein-cannulated, cynomolgus monkeys, following a single oral dose of 30 mg/kg of ³H Ribavirin or (═NH)-modified ³H Ribavirin, peak radioactivity concentrations in RBC were reached after 24 hrs and remained steady thereafter. The peak radioactivity concentrations for ³H Ribavirin and (=N)-modified ³H Ribavirin had a half-life time T_{1/2} of about 1998 hours and 577 hours, respectively. After multiple dosing at 30 mg/kg, steady-state radioactivity concentrations were projected to be significantly higher for Ribavirin as compared to (═NH)-modified ³H Ribavirin (Table 2).

TABLE 2
Differential Radioactivity Distribution of Ribavirin and (=NH) modified
Ribavirin in RBC

| | | ³H-(=NH) |
|---|---|---|
| | ³H-Ribavirin | modified Ribavirin |
| Medium RBC Radioactivity | 1.36 μg equiv/g | 0.38 μg equiv/g |
| (Mice) | | |
| Medium RBC Radioactivity | ˜41 μg equiv/g | ˜17 μg equiv/g |
| (Monkeys - single dose) | | |
| Medium RBC Radioactivity | ˜5089 μg equiv/g | ˜606 μg equiv/g |
| (Monkeys - multiple doses) | | |

The data presented in Table 2 are also in agreement with toxicity findings. Rhesus monkeys receiving 60 mg/kg of Ribavirin, followed by 30 mg/kg*day for 10 days suffered from hemolytic anemia and significant decrease in RBCs. In contrast, monkeys receiving identical doses of modified Ribavirin exhibited no significant changes in RBCs.

Based on the difference between portal plasma and systemic plasma in portal, vein-cannulated monkeys after oral administration of either Ribavirin or modified Ribavirin, the liver radioactivity concentration after oral dosing of modified Ribavirin was estimated to be approximately 50% higher than oral dosing of Ribavirin. Thus, only about 66% of the Ribavirin dosage would be required for modified Ribavirin to achieve the same liver concentration of Ribavirin. Based on the lower RBC radioactivity (˜12%) and higher liver concentration (˜50%) for modified Ribavirin as compared to Ribavirin, the therapeutic ratio for modified Ribavirin is estimated to be about twelve times that of Ribavirin. Therefore, it is contemplated that modified Ribavirin can be administered in a dosage of about 65% of Ribavirin to achieve approximately the same efficacy as Ribavirin with substantially no hemolytic anemia; or that modified Ribavirin can be administered in the same dosage as Ribavirin to achieve higher efficacy as Ribavirin with substantially no hemolytic anemia. It is further contemplated that modified Ribavirin can also be administered in a dosage of only about 5%-50%, preferably 20%-50%, more preferably 10%-1 5%, and most preferably 5-6% of the Ribavirin dosage to achieve the same therapeutic effect as Ribavirin.

(e) In Vitro Deamination of (═NH) Modified Ribavirin to Ribavirin

Adenosine deaminase (ADA) isolated from calf intestine was purchased from Boehringer Mannheim. The assay was performed in Dulbecco's PBS buffer (Na₂HPO₄, 8 mM; KH₂PO₄, 1.5 mM; KCl, 2.7 mM; NaCl, 138 mM; pH 7.2) at room temperature (23° C.). UV spectra of (═NH) modified Ribavirin and Ribavirin (0.2 mM) were obtained and the absorption difference at 240 nm was utilized to follow the hydrolytic deamination of (═NH) modified Ribavirin to Ribavirin. In the absence of enzyme, there is no spontaneous hydrolysis of (═NH) modified Ribavirin observed in the buffer (pH 7.2) for a period of 1.5 h (data not shown), indicating the compound is very stable. Taking the limit of the UV assay method, the spontaneous hydrolysis of viramidine will be less than 2.5×10⁻⁵ min⁻¹. Additional experiments showed that the addition of zinc ion in the buffer does not enhance the spontaneous hydrolysis rate (data not shown).

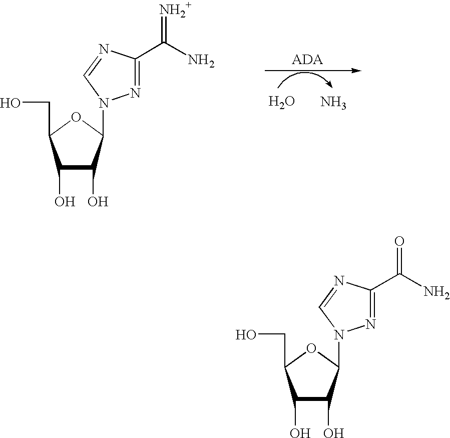

In the presence of 0.2 μM ADA, the deamination of (═NH) modified Ribavirin was accelerated. The enzyme turnover number was estimated about 2.5 min⁻¹ at the current assay condition. Quadruple mass spectrum analysis of the enzyme reaction product indicated that more than 75% of (═NH) modified Ribavirin was converted to Ribavirin after incubating 0.2 mM of (═NH) modified Ribavirin with 0.5 μM ADA overnight.

Thus, specific embodiments and applications of improved specificity in treatments of diseases have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A method of increasing selectivity of a drug with respect to a pharmacological effect, comprising:
identifying a drug as having a desired pharmacological effect on a target cell;
modifying the drug with a blocking group, wherein the blocking group is covalently attached to the drug via a nitrogen atom in the blocking group; and
wherein the blocking group reduces an accumulation of the drug in a non-target cell, and wherein the blocking group is enzymatically removed from the drug in the target cell.

2. The method of claim 1 wherein the drug is selected from the group consisting of a nucleotide, a nucleoside, a nucleotide analog, and a nucleoside analog.

3. The method of claim 2 wherein the drug is 1-beta-D-ribofuranosyl-1,2,4-triazole-3-carboxamide or 2-beta-D-ribofuranosyl-4-thiazolecarboxamide.

4. The method of claim 1 wherein the blocking group comprises at least one of a primary and a secondary amine.

5. The method of claim 1 wherein the blocking group is ═NH.

6. The method of claim 1 wherein the target cell is a hepatocyte.

7. The method of claim 1 wherein the target cell is infected with a virus.

8. The method of claim 1 wherein the target cell is a hyperproliferative cell.

9. The method of claim 1 wherein the accumulation of the drug in the non-target cell comprises phosphorylation of the drug in the non-target cell.

10. The method of claim 1 wherein the non-target cell is an erythrocyte.

11. The method of claim 1 wherein the enzymatic removal of the blocking group from the drug is catalyzed by an aminohydrolase.

12. A method of reducing cytotoxicity of a drug to a non-target cell, comprising:
recognizing that a metabolic conversion of a drug in a non-target cell causes damage to the non-target cell;
modifying the drug with a blocking group, wherein the blocking group is covalently coupled to the drug via a nitrogen atom in the blocking group, and wherein the blocking group reduces the metabolic conversion of the drug in the non-target cell, and is enzymatically cleaved from the drug in the target cell; and
administering the drug to a system comprising the target cell and the non-target cell, wherein the blocking group is covalently coupled to the drug.

13. The method of claim 12 wherein the drug is 1-beta-D-ribofuranosyl-1,2,4-triazole-3-carboxamide, and wherein the metabolic conversion comprises phosphorylation of the drug.

14. The method of claim 12 wherein the non-target cell is an erythrocyte.

15. The method of claim 12 wherein the blocking group is ═NH.

16. The method of claim 12 wherein the damage comprises inhibition of an inosine-5′-monophosphate dehydrogenase.

17. A method of reducing a dosage of a drug in a system comprising a target cell and a non-target cell, comprising:
providing a drug, wherein a metabolic conversion of the drug in a non-target cell reduces a concentration of the drug in a system comprising the non-target cell and a target cell;
modifying the drug with a blocking group, wherein the blocking group is covalently coupled to the drug via a nitrogen atom in the blocking group, and wherein the blocking group reduces the metabolic conversion of the drug in the non-target cell; and
administering the drug to the system, wherein the blocking group is covalently coupled to the drug, and wherein the blocking group is enzymatically removed from the drug in the target cell.

18. The method of claim 17 wherein the system comprises a mammal.

19. The method of claim 17 wherein the drug is 1-beta-D-ribofuranosyl-1,2,4-triazole-3-carboxamide and the blocking group is ═NH.
